SEQUENCE LISTING (7 sequences)

SEQ ID NO: 1 - DNA, 2292 bp, Artificial Sequence
  pEAQ-ACE2-His6-KDEL plasmid
atgggattcgttctcttctctcaacttccatcattcttgttggttagtacattgcttctcttcttggttattagtcattcttgtagagctcagtctactattgaagaacaggctaagacattccttgataagttcaatcatgaggctgaagatctcttctatcaatcttctcttgcttcttggaattacaatacaaatatcacagaggagaacgttcaaaatatgaataatgctggtgataagtggtctgcattcttgaaggaacaatctactcttgctcagatgtaccctcttcaggagattcaaaatcttacagttaagttgcagttgcaagctcttcaacaaaatggttcttctgttcttagtgaagataagtctaagaggcttaatacaattcttaacactatgtctaccatctactctactggaaaggtgtgtaatccagataatccacaggaatgtcttcttcttgagcctggattgaatgagattatggctaattctcttgattacaatgagagattgtgggcttgggagtcttggagatctgaggttggtaagcagcttaggcctctctatgaagaatatgttgttcttaagaacgagatggctagagctaatcattatgaggattatggagattattggagaggtgattacgaagttaatggagttgatggatacgattattctagaggacagttgattgaagatgttgagcatacattcgaagagattaagcctctctatgagcatcttcatgcttatgttagggctaagcttatgaatgcttatccttcttatatctctcctattggttgtcttccagctcatcttcttggagatatgtggggtagattctggactaatctctattctcttactgttccattcggtcagaagcctaatattgatgttactgatgctatggttgatcaggcttgggatgctcagaggatcttcaaggaagctgagaagttcttcgttagtgttggtcttccaaatatgactcaaggattctgggaaaattctatgcttacagatcctggtaatgttcaaaaggctgtgtgtcatcctactgcttgggatcttggaaagggtgacttcaggattcttatgtgtactaaggttactatggatgacttccttacagctcatcatgagatgggtcatattcaatacgatatggcttatgctgctcagccattcttgttgagaaatggtgctaatgagggattccatgaggctgttggagagattatgtctcttagtgctgctactcctaagcatcttaagtctattggattgcttagtcctgacttccaagaagataatgaaactgaaattaacttcctccttaagcaagctcttactattgttggtactcttccattcacttacatgttggaaaagtggagatggatggtgttcaagggtgagattcctaaggatcagtggatgaagaagtggtgggaaatgaagagagagattgttggagttgttgaacctgttcctcatgatgaaacatattgtgatcctgcttctctcttccatgttagtaatgattactcattcatcagatactacacaagaacattgtatcagttccaattccaagaggctctctgtcaagctgctaagcatgaaggtcctcttcataagtgtgatattagtaattctaccgaagctggacaaaagttgttcaatatgcttagattgggaaagtctgaaccttggactcttgctcttgaaaatgttgttggagctaagaatatgaatgttagaccattgttgaactacttcgaacctctcttcacttggttgaaggatcagaataagaattcattcgttggatggtctactgattggtctccatatgctgatcaatctattaaggttaggattagtcttaagtctgctcttggtgataaggcttatgagtggaatgataatgagatgtatctcttcaggtcttctgttgcttacgctatgagacagtacttcttgaaggttaagaatcagatgattctcttcggtgaggaggatgttagagttgctaatcttaagcctaggattagcttcaacttcttcgttactgctcctaagaatgttagtgatattattcctaggacagaagttgaaaaggctattagaatgtctagatctagaattaacgacgcattcaggttgaatgataattctcttgaattccttggtatccagccaactcttggtcctcctaatcaaccaccagttagtcatcatcatcatcaccataaggatgagctttga

SEQ ID NO: 2 - protein, 763 aa, Artificial Sequence
  recombinant protein
MGFVLFSQLPSFLLVSTLLLFLVISHSCRAQSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSHHHHHHKDEL

SEQ ID NO: 3 - protein, 8 aa, Artificial Sequence
  FLAG sequence
DYKDDDDK

SEQ ID NO: 4 - DNA, 942 bp, Artificial Sequence
  recombinant DNA (ENDO H-Flag-KDEL)
atgggtttcgtgctgttcagccagctgccttctttccttcttgtgtctacccttctgctgttcctggtgatctctcattcttgtagggctccagctcctgctcctgttaagcaaggtcctacttctgtggcttacgttgaggtgaacaacaacagcatgctgaacgtgggaaagtacacccttgctgatggtggtggtaacgctttcgatgtggctgtgattttcgctgctaacatcaactacgataccggtactaagaccgcttacctgcacttcaatgagaacgtgcagagggtgttggataacgctgtgactcagattaggcctcttcagcagcagggtattaaggtgctgctttctgtgcttggtaaccaccagggtgctggtttcgctaattttcctagtcagcaggctgcttccgctttcgctaagcaactttctgatgctgtggctaagtacggtctggatggtgtggatttcgatgatgagtacgctgagtacggtaacaacggtactgctcagcctaacgatagctctttcgtgcatcttgtgaccgctctgagggctaacatgcctgataagatcatcagcctttacaacatcggtcctgctgcttccaggctttcttacggtggtgttgatgtgagcgataagttcgattacgcttggaacccttactacggaacctggcaagttcctggtattgctttgcctaaggctcagctttctccagctgctgttgagattggtaggacctctaggtctaccgtggctgatcttgctagaaggactgtggatgagggttacggtgtgtaccttacctacaacctggatggtggtgataggaccgctgatgtgtctgctttcaccagagagctttacggttctgaggctgtgaggacccctgattacaaggacgatgatgataaggatgagctgtag

SEQ ID NO: 5 - protein, 313 aa, Artificial Sequence
  recombinant protein (ENDO H-Flag-KDEL)
MGFVLFSQLPSFLLVSTLLLFLVISHSCRAPAPAPVKQGPTSVAYVEVNNNSMLNVGKYTLADGGGNAFDVAVIFAANINYDTGTKTAYLHFNENVQRVLDNAVTQIRPLQQQGIKVLLSVLGNHQGAGFANFPSQQAASAFAKQLSDAVAKYGLDGVDFDDEYAEYGNNGTAQPNDSSFVHLVTALRANMPDKIISLYNIGPAASRLSYGGVDVSDKFDYAWNPYYGTWQVPGIALPKAQLSPAAVEIGRTSRSTVADLARRTVDEGYGVYLTYNLDGGDRTADVSAFTRELYGSEAVRTPDYKDDDDKDEL

SEQ ID NO: 6 - protein, 4 aa, Artificial Sequence
  ER retention signal
KDEL

SEQ ID NO: 7 - protein, 30 aa, Nicotiana tabacum
  PR-1a signal peptide
MGFVLFSQLPSFLLVSTLLLFLVISHSCRA

(19) United States
(12) Patent Application Publication
(10) Pub. No.: US 2022/0220465 A1
(43) Pub. Date: Jul. 14, 2022

(54) ENGINEERING, PRODUCTION AND CHARACTERIZATION OF PLANT PRODUCED, SOLUBLE HUMAN ANGIOTENSIN CONVERTING ENZYME-2 AS A THERAPEUTIC TARGET IN COVID-19

(71) Applicant: AKDENIZ UNIVERSITESI, Antalya (TR)

(72) Inventor: Tarlan MAMMEDOV, Antalya (TR)

(73) Assignee: AKDENIZ UNIVERSITESI, Antalya (TR)

(21) Appl. No.: 17/534,479

(22) Filed: Nov. 24, 2021

Related U.S. Application Data
(60) Provisional application No. 63/117,487, filed on Nov. 24, 2020.

(51) Int. Cl. C12N 9/48; C12N 9/24; C12N 15/82

(52) U.S. Cl. CPC C12N 9/485; C12Y 304/17023; C12N 15/8205; C12Y 302/01096; C12N 15/8257; C12N 9/2402

(57) ABSTRACT

The present invention relates to materials and methods for engineering, expression and high-level production of cost effective, safe and functional active recombinant truncated human Angiotensin-converting enzyme 2 (ACE2) in plants using transient expression system. In particular, the present invention relates to the production of glycosylated and non-glycosylated forms of ACE2 polypeptide in Nicotiana benthamiana (N. benthamiana) plant. The cost effective, safe and functional active plant produced recombinant ACE2 polypeptides can be used as a potential therapeutic target in COVID-19 patients to block or slow down the virus entering, spread of the virus and protect the lung from injury, also recombinant ACE2 enzymes are used as potential drugs to treat patients by controlling blood pressure.

### TECHNICAL FIELD OF THE INVENTION

The present invention relates to methods of engineering, expression and high-level production of cost effective, safe and functional active recombinant truncated human Angiotensin converting enzyme 2 (ACE2) in plants using a transient expression system. In particular, the present invention relates to the production of glycosylated and non-glycosylated forms of ACE2 polypeptide in Nicotiana benthamiana (N. benthamiana) plant. The cost effective, safe and functional active plant produced recombinant ACE2 polypeptides can be used as a potential therapeutic target in COVID-19 patients to block or slow down the virus entering, spread of the virus and protect the lung from injury. These recombinant ACE2 enzymes can be also used as potential drugs to treat patients by controlling blood pressure.

### BACKGROUND OF THE INVENTION

SARS-CoV-2 is a novel and highly pathogenic coronavirus, which has caused an outbreak in Wuhan city, China in 2019, and then soon spread nationwide and spilled over to other countries and the world. Head of the United Nations has described this as humanity's worst crisis since World War II. Although several vaccines for COVID-19 are present, the efficacy of these vaccines is not fully known. In addition, there are currently no drugs available to protect people against deadly SARS-CoV-2 coronavirus. The world urgently needs an efficient SARS-CoV-2 coronavirus vaccine, antiviral and therapeutic drugs to relieve the human suffering associated with the pandemic that kills thousands of people every day. The development of therapeutic drugs could be also useful approach to inhibit the virus entering and spread.

The receptor binding domain (RBD) in the Spike (S) protein of coronavirus specifically binds to the Angiotensin-converting enzyme 2 (ACE2) receptor on the host cell membrane, and it has been reported that it may be used as a subunit vaccine against coronavirus infection. ACE2 is a zinc containing metalloenzyme, present in most organs, attached to the cell membranes of cells in the lungs, heart, kidney, arteries and intestines. ACE2 enzyme has multiple functions, and its primary function is to cleave the angiotensin I hormone into the vasoconstricting angiotensin II. ACE2 is a transmembrane protein and serves as receptors for some coronaviruses, including SARS-CoV, SARS-CoV-2 and HCoV-NL63 [1, 2, 3, 4, 5]. SARS-CoV has been shown to bind to its functional receptor ACE2 via a spike protein [6]. ACE2 molecule has 7 potential N-glycosylated sites and S-glycoprotein of SARC-CoV-2 has 22 potential N-glycosylation sites. The virus (SARS-CoV-2) and receptor of ACE2 binding affinity on the surface of human cells could be a critical step in viral entry into cells. It has been also demonstrated that ACE2 serves not only the entry receptor for SARS-CoV or SARS-CoV-2 but also can also provide protection from lung injury. Like other respiratory diseases, COVID-19 can cause permanent damage to the lungs, heart and other organs. A possible explanation for this damage is the blocking of the binding domain of the ACE2 receptor by SARC-CoV-2. Therefore, recombinant ACE2 could be a promising target to attenuate or prevent COVID-19 associated cellular injury.

Soluble ACE2 has been described as a therapeutic candidate, which could neutralize the infection by acting as a decoy [7]. It has been suggested that treatment with a soluble form of ACE2 itself may be important to slow down the viral entry into cells and protect the lung from injury [2, 8, 15, 16]. And generally, it has been supposed that soluble form of ACE2 in excessive forms, may negatively affect the virus entering and spreading [17]. Recombinant human ACE2 is also proposed as a novel treatment to improve pulmonary blood flow and oxygen saturation in piglets [18]. Therefore, production of cost effective and enzymatically active recombinant ACE2 is highly demanded.

Numerous studies in recent years have demonstrated plant expression systems and promising expression platforms for cost-effective, fast and safe production of a variety of recombinant proteins. Plant expression systems have several advantages compared to other expression systems that are currently used and have the ability to accumulate hundreds of milligrams of target protein per kilogram of biomass in less than a week. These systems have been successfully used for rapid and cost-effective production of a variety of recombinant proteins, vaccine candidates etc. [9, 19, 10, 11, 12] including vaccines against COVID 19 [14, 20].

In the prior art, human ACE2 was expressed in plant chloroplasts by using transplastomic technology. It was demonstrated that the delivery of human ACE2 (fused with CTB) by oral gavage in mice resulted in increased circulating and retinal levels of ACE2 and reduced eye inflammation (13). However, since expression levels of human ACE2 in plant chloroplasts are not high and considering that ACE2 significantly undergoes to enzymatic digestions in the stomach, this system would have limitations for efficient delivery of high quantity of ACE2 to COVID-19 patients for a short time. ACE2 is a single pass type I membrane protein. Since ACE2 was not isolated from the chloroplast and was expressed as a transmembrane domain, it may not be soluble. There is no evidence whether chloroplast ACE2 is functional active. The chloroplast ACE2 cannot be administered intravenously, intramuscularly or subcutaneously as it is not isolated and purified.

ACE2 enzyme were also produced in various mammalian cells (HEK293, CHO, insect cells etc.). However, mammalian expression systems are extremely expensive and difficult to scale up. In addition, there is a risk of contamination of mammalian pathogens in recombinant proteins produced using the mammalian expression systems.

According to the problems in the prior art such as low expression levels of human ACE2 in plant chloroplasts, low production levels of ACE2 with known methods, extremely expensive expression systems for the production of ACE2 and risk of contamination of mammalian pathogens in recombinant proteins produced using the mammalian expression systems; developments in the method for generating high-level production of cost effective, safe and functional active recombinant human ACE2 polypeptide in plants is needed in this technical field.

### SUMMARY OF THE INVENTION

The present invention discloses a functional active recombinant truncated human Angiotensin-converting enzyme 2 (ACE2) and the methods for modifying, expressing and producing said ACE2 in high levels by using transient expression system in plants. Engineering and modifying ACE2 makes it possible to produce the ACE2 enzyme at high levels in plants. In the present invention, a high-level production (about ˜0.75 g/kg leaf biomass) of human soluble ACE2 in Nicotiana benthamiana (N. benthamiana) plant and high purification yields of recombinant plant produced ACE2 protein in glycosylated and deglycosylated forms (˜0.4 and 0.5 g/kg leaf biomass, respectively) are provided.

The aim of this invention is to provide a method to produce high levels of ACE2 enzyme with a cost-effective manner. The expression and production of soluble human ACE2 has not been previously reported in plant systems in the prior art. In the present invention, ACE2 enzyme is engineered/modified to provide a high level of active recombinant human ACE2 enzyme production in N. benthamian plant. Plant expression systems have a number of advantages compared to other expression systems that are currently used and these systems have the ability to accumulate hundreds of milligrams of target protein per kilogram of biomass in less than a week. In the present invention, plant expression system in N. benthamian plant has been successfully used for rapid and cost-effective production of a variety of ACE2 recombinant proteins. The purification yield of recombinant plant produced ACE2 protein (glycosylated and deglycosylated) is calculated as ˜0.5/kg leaf biomass, respectively. Expression level and purification procedure can be optimized to increase the purification yield by different ways. For example, purification yield can be increased by agrobacterium optimization, by using different agro bacterium strains or by plant Rubisco protein removal from total extract etc. The purity of said recombinant plant produced ACE2 protein in the present invention is higher than 90%.

Another aim of the invention is to provide antiviral drugs and safe candidates as a potential therapeutic comprising recombinant ACE2 enzyme for use in the treatment of COVID-19. In the present invention, both glycosylated and non-glycosylated variants of recombinant ACE2 protein in N. benthamiana plant is produced to understand the role of glycosylation. In the invention, deglycosylated ACE2 variant is produced by using the in vivo deglycosylation technology, by co-expression of ACE2 with bacterial Endo-β-N-acetylglucosaminidase H (Endo H) (10). As shown in the present invention, plant produced glycosylated and non-glycosylated ACE2s are active and successfully bind to spike protein of SARC-CoV-2. However, the deglycosylated ACE2 variant binds to the deglycosylated plant-produced S-protein much more strongly than the glycosylated counterparts. In the present invention, the plant produced recombinant ACE2 is used as a potential therapeutic target in COVID-19 patients to block and slow down the virus entering and spread of the virus and to protect the lung from injury. It is known in the prior art that ACE2 in excessive forms can slow down the virus entering, spread of the virus and protect the lung from injury. In the present invention, the development of production of cost effective, safe and functional active recombinant ACE2 is provided and this recombinant ACE2 enzyme is used in the treatment of COVID-19 patients.

Plant produced, safe and cost effective recombinant ACE2 enzymes explained in the invention are also used as potential drugs to treat patients by controlling blood pressure. In the present invention, ACE2 enzyme solutions can be administered by inhalation, preferably using a concentration of 0.1-1.0 mg/ml. ACE2 enzymes can be administered orally (tablet, etc.) or injected intramuscularly (intramuscularly) or subcutaneously (subcutaneously). ACE2 enzymes can also be used as nasal spray to block the virus entering.

Another aim of the invention is to provide a stable recombinant ACE2 enzyme that successfully and strongly binds to the SARS-CoV-2 spike protein. In the present invention, glycosylated and deglycosylated forms of recombinant ACE2 enzyme is produced. The recombinant human soluble ACE2 that is produced by plant expression system is shown that it successfully binds to the SARS-CoV-2 spike protein. However, the deglycosylated ACE2 variant binds to the deglycosylated plant-produced S-protein much more strongly than the glycosylated counterparts. Importantly in the present invention, both deglycosylated and glycosylated forms of ACE2 are stable at elevated temperatures for extended periods of time and these two forms demonstrated strong anti-SARS-CoV-2 activities in vitro. The IC50 values of glycosylated and deglycosylated ACE2 were 1.020 and 1.342 μg/ml, respectively, for the pre-entry infection, when incubated with 100TCID₅₀ of SARS-CoV-2. Therefore, plant produced soluble ACE2s are considered as promising cost-effective and safe candidates as a potential therapeutic tool in the treatment of COVID-19 patients. In particular, deglycosylated plant produced ACE2 is a more promising candidate as a potential therapeutic target in COVID-19 patients.

Given the high morbidity and mortality rates, which associated with COVID-19, there is an urgent demand for developing effective, cost effective and safe therapeutics, vaccines and inhibitors to control the epidemic. The present invention overcomes the problems which are low expression levels of human ACE2 in plant chloroplasts, low production levels of ACE2 with known methods, extremely expensive expression systems for the production of ACE2, risk of contamination of mammalian pathogens in recombinant proteins produced using the mammalian expression systems, inadequacy of current drugs and vaccines for COVID-19 treatment and prevention and other disadvantages present in the prior art by providing a method for generating high-level production of cost effective, safe and functional active recombinant human ACE2 in plants, and therefore providing antiviral drugs and safe candidates as a potential therapeutic comprising recombinant ACE2 polypeptide for use in the treatment of COVID-19.

### BRIEF DESCRIPTION OF THE DRAWINGS

FIG. 1. Western blot analysis of human ACE2s, produced in N. benthamiana plants. (dACE2 (deglycosylated): human ACE2 co-expressed with bacterial Endo H, produced in N. benthamiana, different concentration (dilutions) of crude extract; gACE2 (glycosylated): human ACE2, produced in N. benthamiana, different concentration (dilutions) of crude extract; C—undiluted crude extract from non-infiltrated N. benthamiana; gPA83: 25, 50, 100 ng of purified plant produced dPA83 of Bacillus anthracis, as a control protein to quantify the expression levels of ACE2 and ACE2 proteins.)

FIG. 2. SDS-PAGE (A) and Western blot (B) analysis of plant produced, Ni-NTA resin purified glycosylated or deglycoslated ACE2 proteins. (gACE2: 5 or 10 μg purified glycosylated ACE2 protein; dACE2: 5 or 10 μg purified deglycosylated ACE2 proteins. BSA standards: 1.0, 2.5 and 5.0 μg BSA protein as a standard protein. B: membrane probed with anti-His6 antibody. gPA83 (plant produced glycosylated protective antigen of Bacillus anthracis, MM ˜100 kDa) and dPA83 (deglycosylated protective antigen of Bacillus anthracis, MM ˜90 kDa) proteins used as a standard. C: membrane probed with a purified anti-human ACE2 antibody.)

FIG. 3. Gel filtration chromatography (A) and SDS-PAGE (B) of plant-produced gACE2 or dACE2 proteins. ((A) Profiles of BSA, plant-produced gACE2, dACE2 and PA83 proteins. (B) SDS-PAGE analysis of plant-produced gACE2 and dACE2 proteins.)

FIG. 4. Binding activity of plant produced, glycosylated or deglycosylated variants of ACE2 with commercial or plant produced, glycosylated or deglycosylated forms of spike proteins (Flag tagged). (Com S: commercial Spike protein, active Recombinant 2019-nCoV Spike Protein, RBD, His Tag, produced in Baculovirus-Insect Cells; pp-gRBD: plant produced glycosylated Receptor Binding Domain of Spike protein; pp-dRBD: plant produced deglycosylated RBD; pp-gACE2: plant produced glycosylated ACE2; pp-dACE2: plant produced Endo H in vivo deglycosylated ACE2; Endo H, plant produced Flag-tagged protein as negative control. A, B: graph for binding affinity between pp-gACE2 and pp-dACE2 to spike protein variants.)

FIG. 5. Stability assessment of plant produced glycosylated and deglycosylated ACE2 proteins. (A: Plant produced, Ni-NTA resin column purified gACE2 or dACE variants incubated at 37° C. for 24, 48, 72, 96, 120 and 144 hours, and analyzed in SDS-PAGE. B: Plant produced, Ni-NTA resin column purified gACE2 or dACE variants incubated at 72 and 144 hours, and different amount (0.5, 1.0 and 2.0 μg) from each sample, analyzed in SDS-PAGE; M: color prestained protein standard.)

FIG. 6. Binding affinity of plant produced glycosylated and deglycosylated ACE2 proteins after incubation at 37° C. for 72 or 144 hours.

FIG. 7. Apparent activities of two distinct ACE2 (glycosylated and deglycosylated forms of ACE2) derivatives produced in plants to RBDs plotted against IC₅₀ of authentic SARS-CoV-2 neutralization. (gACE2: glycosylated ACE2; dACE2: plant produced deglycosylated ACE2 (IC₅₀ dACE2=1.342, IC₅₀ ACE2=1.020)).

### DETAILED DESCRIPTION OF THE INVENTION

The present invention provides materials and methods for modification, expression and high-level production of cost effective, safe and functional active recombinant truncated human Angiotensin-converting enzyme 2 (ACE2) in plants using transient expression system. In particular, the production of glycosylated and non-glycosylated forms of ACE2 polypeptide in Nicotiana benthamiana (N. benthamiana) plant is provided in the present invention.

The subject matter of the invention discloses the method for generating ACE2 polypeptide in N. benthamiana plants which comprises cloning, expression, screening and purification of recombinant ACE2 in N. benthamiana plants, and also obtaining the binding affinity of recombinant ACE2 to RBD and obtaining recombinant ACE2's SARS-CoV-2 virus neutralizing ability. Within the scope of the invention, after obtaining the said recombinant ACE2 polypeptides, binding affinity of plant produced recombinant ACE2 protein with spike protein is determined. Stability assessments of different variants of ACE2 are performed and anti-SARS-CoV2 activity of plant produced ACE2s is evaluated.

In the present invention, two embodiments, glycosylated and non-glycosylated forms of ACE2 polypeptide in N. benthamiana plant is provided. Methods for generating glycosylated human ACE2 gene (gACE2) and deglycosylated human ACE2 gene (dACE2) differs only in the step of co-expression. Other method steps are the same in gACE2 and dACE2. The only difference between these two embodiments is that for the expression of dACE2, ACE2 gene is in vivo co-expressed with Endo H gene.

In the first embodiment of the present invention, a recombinant version of glycosylated human ACE2 (truncated) in N. benthamiana plant is produced. Cloning, expression, and screening of recombinant ACE2 in N. benthamiana plants is performed. The sequences of ACE2 (without a transmembrane domain and cytoplasmic tail) were optimized for expression in N. benthamiana plants and synthesized by Biomatik (Biomatik corporation). To express ACE2 in N. benthamiana plants, the signal peptide of human ACE2 (amino acids 1-17) was replaced with the Nicotiana tabacum PR-1a signal peptide having amino acid sequence of SEQ ID NO.7. In addition, the ER retention signal having amino acid sequence of SEQ ID NO.6 and the His6 tag coding sequences were added to the C-terminus and artificial ACE2 gene is constructed. The constructed ACE2 gene was inserted into the pEAQ binary expression vector to obtain pEAQ-ACE2-His6-KDEL plasmid having a nucleic acid construct that has at least 90 percent sequence identity to the sequence of SEQ ID NO:1, preferably having nucleotide sequence of SEQ ID NO.1. Then, pEAQ-ACE2-His6-KDEL plasmid preferably having nucleotide sequence of SEQ ID NO.1 was introduced into an Agrobacterium construct, preferably Agrobacterium tumefaciens strain AGL1. Agrobacterium construct carrying the pEAQ-ACE2-His6-KDEL plasmid was then infiltrated into 6-7-week-old N. benthamiana plants. In the present invention, the nucleotide sequences that have at least 90 percent sequence identity to the sequence of Seq ID NO.1 and Seq ID NO.4 can also be used since 90% identity provides the same results.

The method for generating a polypeptide of glycosylated ACE2 in a plant cell is explained step by step below, said method comprises the steps of:
replacing signal peptide of human ACE2 with Nicotiana tabacum PR-1a signal peptide having amino acid sequence of SEQ ID NO.7, adding ER retention signal having amino acid sequence of SEQ ID NO.6 and adding His6 tag coding sequence to C-terminus and constructing an artificial ACE2 gene; wherein the artificial ACE2 gene is operable linked to a promoter such that when the promoter is activated, the ACE2 polypeptide is expressed,
inserting the constructed ACE2 gene into small binary vector tailored for transient expression (pEAQ vector) to obtain pEAQ-ACE2-His6-KDEL plasmid having a nucleotide sequence that has at least 90 percent sequence identity to sequence of SEQ ID NO:1,
introducing pEAQ-ACE2-His6-KDEL plasmid having a nucleotide sequence that has at least 90 percent sequence identity to sequence of SEQ ID NO:1 into the Agrobacterium construct, preferably the Agrobacterium tumefaciens strain AGL1,
performing infiltration of the Agrobacterium construct, preferably the Agrobacterium tumefaciens strain AGL1 carrying the pEAQ-ACE2-His6-KDEL plasmid having a nucleotide sequence that has at least 90 percent sequence identity to sequence of SEQ ID NO:1 into plant cell, preferably 6-7-week-old N. benthamiana plant leaf cell, and producing a polypeptide of glycosylated ACE2 having amino acid sequence of SEQ ID NO.2.

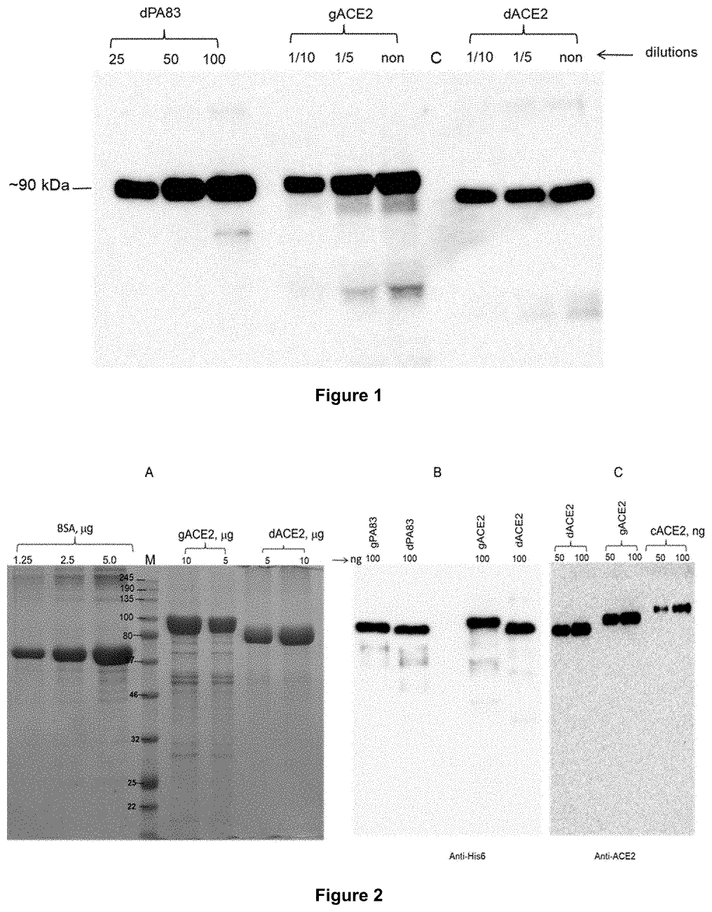

In the second embodiment of the present invention, a recombinant version of deglycosylated human ACE2 (truncated) in N. benthamiana plant is produced. Cloning, expression, and screening of recombinant ACE2 in N. benthamiana plants is performed. To confirm the expression of His6 tagged ACE2 protein variants, a leaf tissue was harvested at different dpi (day post infiltration) and homogenized in three volumes of extraction buffer (20 mM sodium phosphate, 150 mM sodium chloride, pH 7.4). For deglycosylated ACE2 production, ACE2 gene was produced by using the in vivo deglycosylation technology, co-expression of ACE2 with bacterial Endo-β-N-acetylglucosaminidase H (Endo H). To confirm the expression of His6 tagged ACE2 protein variants, a leaf tissue was harvested at 6 dpi (day post infiltration) and homogenized in three volumes of extraction buffer (20 mM sodium phosphate, 150 mM sodium chloride, pH 7.4). Agrobacterium growth, plant growth, plant infiltration, plant leaf tissue harvesting, extraction, homogenization and further analysis were performed as described in prior art. In FIG. 1, western blot analysis of human ACE2s, produced in N. benthamiana plants is shown, purified anti-His Tag antibody (Cat. No. 652502, BioLegend) was used as a primary and mouse IgG used as secondary antibodies to detect ACE2 proteins. As shown in FIG. 1 that demonstrates Western blot analysis of human ACE2s, produced in N. benthamiana plants; the expression level of gACE2 and dACE2 proteins in N. benthamiana plant are calculated.

The method for generating a polypeptide of N-deglycosylated ACE2 in a plant cell is explained step by step below, said method comprises the steps of:
replacing signal peptide of human ACE2 with Nicotiana tabacum PR-1a signal peptide having amino acid sequence of SEQ ID NO.7, adding ER retention signal having amino acid sequence of SEQ ID NO.6 and adding His6 tag coding sequence to C-terminus and constructing an artificial ACE2 gene,
inserting the constructed ACE2 gene into small binary vector tailored for transient expression (pEAQ vector) to obtain pEAQ-ACE2-His6-KDEL plasmid having a nucleotide sequence that has at least 90 percent sequence identity to sequence of SEQ ID NO:1; wherein the artificial ACE2 gene is operable linked to a promoter such that when the promoter is activated, the ACE2 polypeptide having amino sequence of SEQ ID NO.2 is expressed,
separately from pEAQ-ACE2-His6-KDEL plasmid, constructing an ENDO H-Flag-KDEL plasmid having nucleotide sequence of SEQ ID NO.4 by adding a second nucleic acid encoding a bacterial Endo-β-N-acetylglucosaminidase H (Endo H), adding ER retention signal having amino acid sequence of SEQ ID NO.6 and adding Flag tag coding sequence; wherein the Endo H sequence is operable linked to a promoter such that when the promoter is activated, the Endo H polypeptide having amino acid sequence of SEQ ID NO.5 is expressed,
introducing pEAQ-ACE2-His6-KDEL plasmid having a nucleotide sequence that has at least 90 percent sequence identity to sequence of SEQ ID NO:1 into a Agrobacterium construct, preferably the Agrobacterium tumefaciens strain AGL1,
introducing ENDO H-Flag-KDEL plasmid having nucleotide sequence of SEQ ID NO.4 into another Agrobacterium construct, preferably the Agrobacterium tumefaciens strain AGL1,
performing co-infiltration of Agrobacterium tumefaciens strain AGL1 carrying the pEAQ-ACE2-His6-KDEL plasmid having a nucleotide sequence that has at least 90 percent sequence identity to sequence of SEQ ID NO:1 with Agrobacterium tumefaciens strain AGL1 containing ENDO H-Flag-KDEL plasmid having nucleotide sequence of SEQ ID NO.4 into plant cell, preferably 6-7-week-old N. benthamiana plant leaf cell, and producing a polypeptide of deglycosylated ACE2 having amino acid sequence of SEQ ID NO.2, wherein by action of the Endo H polypeptide having amino acid sequence of SEQ ID NO.5, ACE2 is deglycosylated with no amino acid change in the asparagine-X-serine/threonine (NXS/T) site (NXS/T motif is the consensus motif for N-linked glycosylation), wherein X is any amino acid except proline of resulting 15 polypeptide, opposite to that of action of the bacterial PNGase F, which causes amino acid change in the deglycosylated protein targets due to deamidation of the asparagine (N) in the NXS/T site (sequence) into an aspartate (D).

PNGase F is a 34.8-kDa enzyme secreted by a gram-negative bacterium Flavobacterium meningosepticum that cleaves a bond between the innermost GlcNAc and asparagine residues of high-mannose, hybrid and complex oligosaccharides in N-linked glycoproteins, except when the a (1-3) core is fucosylated.

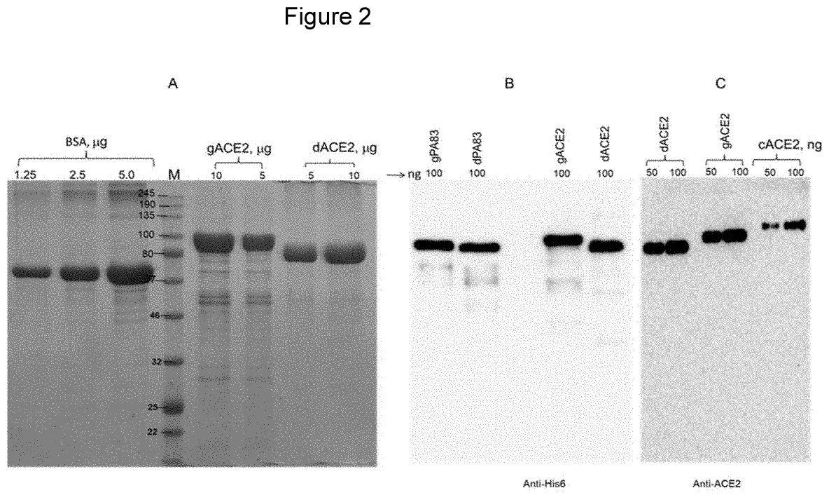

In the step of purification of recombinant ACE2 from N. benthamiana plants, to produce the ACE2 protein (both glycosylated and deglycosylated variants) in N. benthamiana, plants were infiltrated with ACE2 (glycosylated) or ACE2+Endo H (deglycosylated) genes and harvested at 6 dpi. For purification, 20 grams of frozen plant leaves from each variant, infiltrated with the ACE2 gene, were ground in an extraction buffer with a 3 times volume of plant weight and the extract was centrifugated for 20 minutes at 4° C. at 13,000 g. The supernatant was loaded onto a disposable polypropylene column (Pierce) with 1 ml HisPur™ nickel-nitrilotriacetic acid (Ni-NTA) resin equilibrated with 10 column volume binding buffer (20 mM sodium phosphate, 300 mM sodium chloride, 10 mM imidazole, pH 7.4), by gravity-flow chromatography. The column was washed with 10-15 column volumes (CV) of wash buffer ((20 mM sodium phosphate, 300 mM sodium chloride, 25 mM imidazole; pH 7.4) until reaching to the baseline. Proteins were eluted with 10 CV of elution buffer (20 mM sodium phosphate, 300 mM sodium chloride, 250 mM imidazole; pH 7.4). Elution fractions were collected as 0.5 ml/eppendorf and protein concentrations in the eluted fractions were measured by BioDrop. According to the concentration, the combined fractions were concentrated, and buffer exchanged against PBS with a 10K MWCO Millipore concentrator (Cat No: UFC801096, Merck) to a final volume of 1.2 ml. The concentrated protein was stored at (−80)° C. until use. In FIG. 2 SDS-PAGE (A) and Western blot (B) analysis of plant produced, Ni-NTA resin purified glycosylated or deglycoslated ACE2 proteins are shown, glycosylated and deglycosylated plant produced ACE2 proteins were purified from N. benthamiana plant using HisPur™ Ni-NTA resin. The image was taken using a highly sensitive GeneGnome XRQ Chemiluminescence imaging system. As can be seen from FIG. 2 that demonstrates the SDD-PAGE analysis of purified ACE2 proteins, purity and purification yield of plant produced ACE2 proteins are calculated.

After obtaining said recombinant ACE2 protein having amino acid sequence of SEQ ID NO.2, in the step of determining the binding activity of plant produced recombinant ACE2 proteins with commercial or plant produced RBD of spike proteins of SARS-CoV-2, ELISA was performed. Briefly a 96-well plate (Greiner Bio-One GmbH, Germany) was coated with 100 ng of plant produced RBD (R319-S591) or commercial insect RBD of SARS-CoV-2 (RBD, His Tag, Arg319-Phe541, MM˜25 kDa, MBS2563882, MyBioSource, USA) in 100 mM carbonate buffer for overnight. The next day, wells were blocked with blocking buffer (0.5% I-block in PBS) for 2 hours at room temperature. After blocking, various concentrations of plant produced glycosylated and deglycosylated ACE2 proteins (100-2000 ng) were added into wells and incubated for 2 hours at 37° C. After 2 hours, purified anti-His tag mouse mAb (Cat. no. 652505, BioLegend) or purified anti-human ACE2 Antibody (Cat. no. 375801, BioLegend) was added into each well. The plate was washed three times with blocking solution (200 μl/well). After washing, wells were incubated with anti-mouse HRP-IgG antibody (Cat. no. 405306, BioLegend) or anti-human IgG+HRP antibody (Cat. no. MBS440121). The plate was washed three times with washing solution (200 μl/well for 5 minute). 200 μl of substrate solution (Sigma) was added to each well. Afterwards the plate was incubated in the dark, for 30 minutes at room temperature. After the incubation period, the plate was read at 450 nm on a multi-well plate reader.

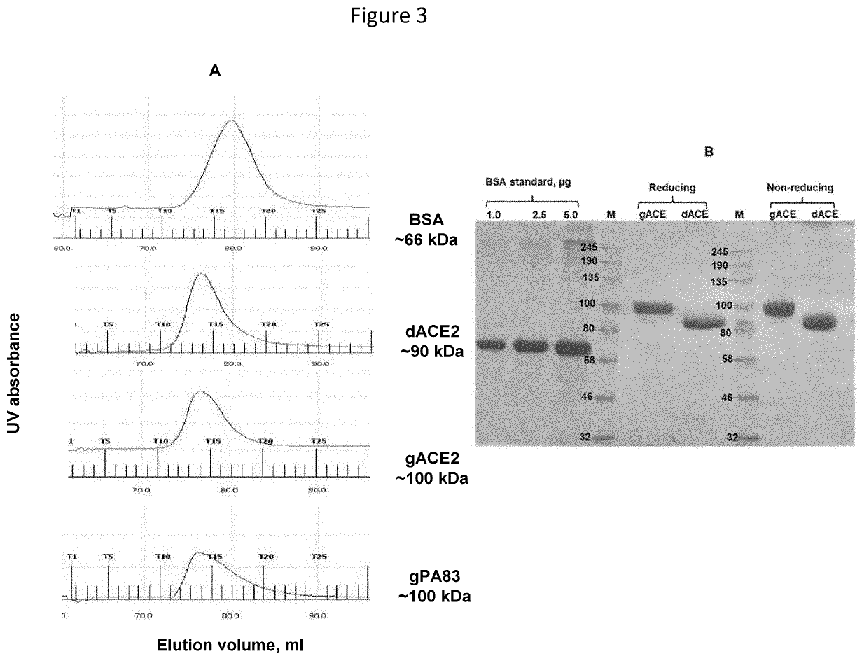

FIG. 3 demonstrates gel filtration chromatography (A) and SDS-PAGE (B) of plant-produced gACE2 or dACE2 proteins, eluted from Sephacryl® S-200 HR column. Both gACE2 and dACE2 were eluted as single picks from Sephacryl S-200 column (FIG. 3A), with elution volumes of 15.62 ml and 15.86 ml, respectively, and were present as monomers (FIG. 3A) as eluted between gPA83 (monomer, ˜90 kDa) and BSA (monomer, ˜66 kDa). No dimerization or aggregation was observed for plant produced gACE2 and dACE2 proteins (FIG. 3B). The column was equilibrated with 50 mM phosphate buffer (with 150 mM NaCl, pH 7.4). BSA, plant-produced dACE2, gACE2 and gPA83 proteins, purified using His-tag affinity chromatography, were loaded onto columns. Gel filtration was performed with AKTA start using C 10/40 column (cat. no. 19-5003-01, GE Healthcare, Chicago, Ill., USA), packed with Sephacryl® S-200 HR (cat. no. 17-0584-10, GE Healthcare). gPA8: plant produced, glycosylated PA83 of Bacillus anthracis, produced in the laboratory. In B section, SDS-PAGE analysis of plant-produced gACE2 and dACE2 proteins are shown, in reduced and non-reducing conditions as indicated. Lanes were loaded with 2.5 μg gACE2 or dACE2.

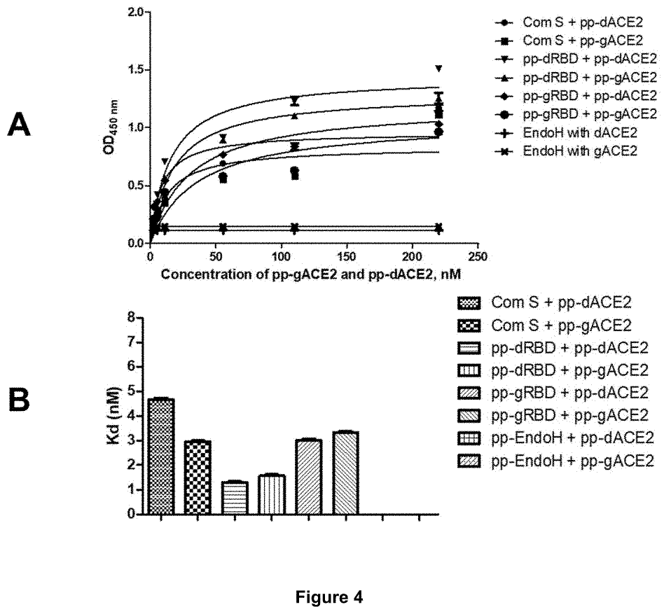

FIG. 4 demonstrates binding activity of plant produced glycosylated or deglycosylated variants of ACE2 with commercial or plant produced, glycosylated or deglycosylated forms of spike proteins (Flag tagged). In FIG. 4, commercial or plant-produced spike protein was coated with an ELISA plate at a concentration of 200 ng/well. Different concentration of plant produced ACE2 (his tagged) was added. Purified anti-His Tag antibody (Cat. No. 652502, BioLegend) was used as a primary and mouse IgG used as secondary antibodies. Com S: commercial Spike protein, active Recombinant 2019-nCoV Spike Protein, RBD, His Tag, produced in Baculovirus-Insect Cells, Cat: MBS2563882); pp-gRBD: plant produced glycosylated Receptor Binding Domain of Spike protein; pp-dRBD: plant produced deglycosylated RBD; pp-gACE2: plant produced glycosylated ACE2; pp-dACE2: plant produced Endo H in vivo deglycosylated ACE2; Endo H, plant produced Flag-tagged protein was used as negative control. A, B: graph for binding affinity between pp-gACE2 and pp-dACE2 to spike protein variants. A: graph was plotted with non-linear regression analysis in Graphpad software. Points refers to absorbance for each sample dilutions and lines were plotted according to Kd value. B: Column bar graph of Kd values determined with non-linear regression analysis in Graphpad software.

The results presented in FIG. 4 demonstrate that plant produced glycosylated and deglycosylated ACE2s successfully bind to commercial spike protein or plant produced RBD of spike protein of SARS-CoV-2. Kd (equilibrium dissociation constant) values (FIG. 4B) ranged from 1.287±0,0317 nM (plant produced dRBD and plant produced dACE2) to 4.678±0.0367 nM (corn S and plant produced dACE2), and a comparable stronger binding effect was observed between plant produced dRBD and dACE2 proteins (1.287±0.0317 nM). This Kd value, determined by ELISA in this study is comparable to Kd reported for hACE2-Spike protein of SARS-CoV-2 (1.2±0.1), determined using Blitz (Walls et al., 2020). Notably, SARS-CoV-2-RBD binding to hACE2, determined by ELISA was reported to be 5.09 nM (Yi et al., 2020), which is comparable to Kd determined using Blitz, 2.9 nM.

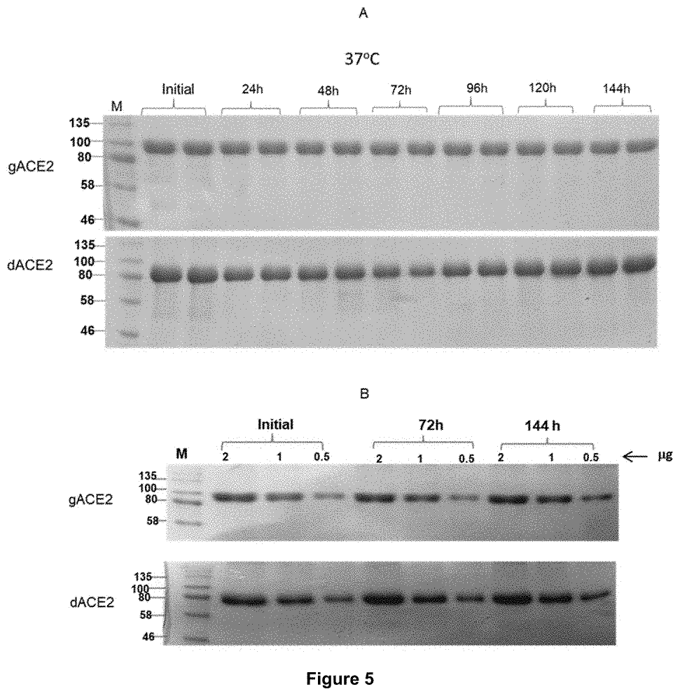

In FIG. 5A, plant produced, Ni-NTA resin column purified gACE2 or dACE variants incubated at 37° C. for 24, 48, 72, 96, 120 and 144 hours, and analyzed in SDS-PAGE. Lanes were loaded with 5.0 μg gACE2 or dACE2. In B, plant produced, Ni-NTA resin column purified gACE2 or dACE variants were incubated at 72 and 144 hours, and different amount (0.5, 1.0 and 2.0 μg) from each sample were analyzed in SDS-PAGE M: color prestained protein standard. FIG. 5 demonstrates stability assessment of plant produced glycosylated and deglycosylated ACE2 proteins. Analysis by SDS-PAGE showed that plant produced glycosylated ACE2 had almost no degradation at 37° C. for 144 hours and degradation of in vivo Endo H deglycosylated ACE2 at the same condition was less than 5%.

Stability assessments of different variants of ACE2 were also performed using a similar procedure as described in prior art. Plant produced glycosylated and deglycosylated variants of ACE2 were diluted to 1.0 mg/mL with PBS and were aliquoted into low-binding tubes. Proteins were then incubated at 37° C. for 24, 48, 72, 96, 120 and 144 hours. After incubation, samples were analyzed by SDS-PAGE and ELISA. For SDS-PAGE analysis, the samples were mixed with SDS loading dye (5×) and stored at −20 ° C. until use. All samples were then run on SDS-PAGE. The degradation of ACE2 variants were quantified using highly sensitive Gene Tools software (Syngene Bioimaging, UK) and ImageJ software (https://imagej.nih.gov/ij). Plant produced gACE2 or dACE2 (ACE2 co-expressed with bacterial Endo H, produced in N. benthamiana, different concentration (dilutions) of crude extract) proteins, which were incubated at 37° C. for 72 or 144 hours were used for ELISA to analyze their binding affinity to commercial S protein (Com S) or plant produced dRBD.

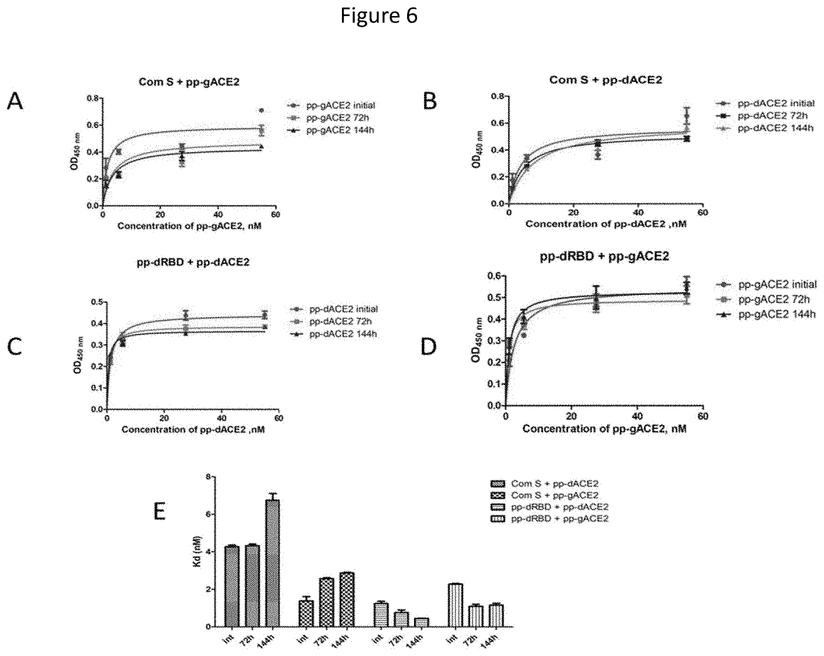

In FIG. 6, binding affinity of plant produced glycosylated and deglycosylated ACE2 proteins are shown. Plant produced gACE2 or dACE2 proteins incubated at 37° C. for 72 or 144 hours were used for ELISA to analyze binding affinity to commercial S-protein (Corn S) or dRBD. A, B, C and D graphs was plotted with non-linear regression analysis in Graphpad software. Points refers to absorbance for each sample dilutions and lines were plotted according to Kd value. In E graph, column bar graph of Kd values determined with non-linear regression analysis in Graphpad software.

FIG. 6 demonstrates the binding affinity of plant produced glycosylated and deglycosylated ACE2 proteins after incubation at 37° C. for 72 and 144 hours. Although the binding affinity of gACE2 and dACE2 proteins that were incubated at 37° C. for 72 or 144 hours was reduced for the commercial Spike protein, it did not change significantly for plant-produced dRBD.

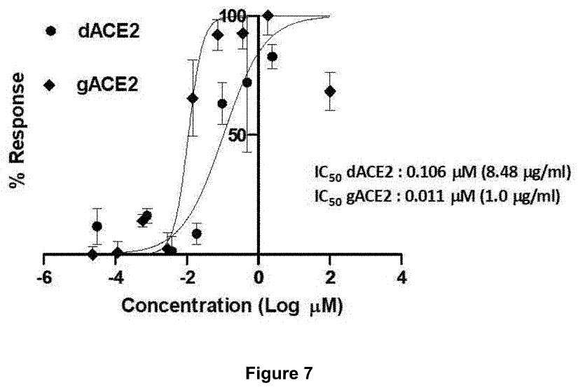

In FIG. 7, IC₅₀ values of the ACE2 (glycosylated) and dACE2 (deglycosylated) were calculated using normalized optical density data obtained from quadruplicated test dilutions in GraphPad Prism v8.2 software (GraphPad). Optical density values from untreated (cell control) wells were used as normalization standards. Nonlinear regression analysis was performed using log (inhibitor) versus normalized response-variable slope. The R square values were recorded as 0.6581 and 0.9581 for dACE2 and ACE2, respectively. FIG. 7 demonstrates apparent neutralization activities of plant produced recombinant truncated gACE2 and dACE2 variants against authentic SARS-CoV-2 in the pre-infection phase. The half maximal inhibitory concentration (IC50) values for glycosylated and deglycosylated ACE2 were ˜1.00 μg/ml (0.011 μM) and 8.48 μg/ml (0.106 μM), respectively, when they were mixed with 100TCID50 of SARS-CoV-2

Anti-SARS-CoV2 activity of plant produced ACE2s is also determined and anti-SARS-CoV-2 potential of ACE2 derivates was monitored in vitro. To do this, blocking capacity of plant produced gACE2 or dACE2 variants at different concentrations are analyzed. Purified dACE2 and gACE2 (initial concentrations were 3,055 and 2,542 mg/mL, respectively) were 5-fold diluted in high glucose DMEM in a U-bottomed plate. After being combined with an equal volume (100 μL) of 100TCID50 virus, the mixtures were incubated at room temperature for 30 minutes. A total of 150 μl incubated mixture was then inoculated on Vero E6 Cells grown in a 96-well flat-bottomed tissue culture plate (Greiner, Germany). The highest concentration (6 μg/ml) of dACE2 and gACE2 without the virus was involved as a toxicity control, and serum-free high glucose DMEM was added to each plate as a cell control. A total of 75 μL 100TCID₅₀ SARS-CoV2 Ank1 virus was also used as virus control. All tests were performed in a quadruplicate. The plates were incubated at 37° C. in a humidified incubator with a 5% CO₂ atmosphere until virus control wells had adequate cytopathic effect (CPE) readings. The test was evaluated when the virus control wells showed 100% CPE at daily microscopy. To do precise calculations based on OD values, cells were fixed with 10% formaldehyde for 30 minutes and subsequently stained with crystal violet (CV −0.075% in ethanol) for 20 minutes. The dye washed away by repeated washing and retained CV was released by adding 100 μL ethanol (70%). Ten minutes after, the plate was read on ELISA reader using 295 nm filter (Multiskan Plus, MKII, Finland).

In the present invention truncated versions of human ACE2 in N. benthamiana plant is produced. Both glycosylated and de-glycosylated variants of ACE2 protein in N. benthamiana plant are produced to understand the role of glycosylation. FIG. 1 demonstrates the confirmation of the production of glycosylated and de-glycosylated variants of ACE2 in N. benthamiana by western blot analysis. N. benthamiana leaf samples were harvested at different post infiltration days (dpi) and expression levels of glycosylated and de-glycosylated variants of ACE2 reached the maximum level at 6 dpi. For purification, a vacuum infiltration was used for large-scale production of glycosylated and de-glycosylated variants of ACE2. Glycosylated and deglycosylated variants of ACE2 were purified using HisPur™ Ni-NTA resin. The purification yields of recombinant plant produced glycosylated or deglycosylated forms were ˜0.4 and ˜0.5 g/kg of leaves, respectively. The purity of glycosylated and deglycosylated variants of ACE2 enzyme was higher than 90% or 95%, for glycosylated or deglycosylated, respectively, as estimated based on SDS-PAGE (FIG. 2A, using BSA a standard protein) and western blot analysis (FIG. 2B, using plant produced, purified deglycosylated PA83 as a standard protein) (FIG. 2). Based SDS-PAGE, under reducing condition, molecular masses were 80 and 90 kDa for deglycosylated and glycosylated ACE2, respectively (FIG. 2).

The binding activity of plant produced recombinant ACE2 protein having amino acid sequence of SEQ ID NO.2 was confirmed by measuring the binding activity of ACE2 with commercially available spike protein or plant produced RBD of spike protein of SARS-CoV-2. The results presented at FIG. 4 demonstrate that plant produced glycosylated and de-glycosylated ACE2s successfully bind to commercial Spike protein or plant produced RBD of spike protein of SARS-CoV-2. Kd (equilibrium dissociation constant) values ranged from 1.217±0.056 nM (plant produced dRBD and plant produced dACE2) to 4.558±0.266 nM (corn S and plant produced dACE2), and a comparable stronger binding effect was observed between plant produced dRBD and dACE2 proteins (1.217±0.056 nM). Notably, SARS-CoV-2-RBD binding to hACE2, determined by ELISA was reported to be 5.09 nM in the prior art, which is comparable to Kd determined using Blitz, 2.9 nM or 1.2±0.1 nM.

The stability of plant produced glycosylated and in vivo deglycosylated forms of ACE2 were examined after incubation at 37° C. for a prolonged time period: 24, 48, 72, 96, 120 and 144 hours (FIG. 5). Analysis by SDS-PAGE showed that plant produced glycosylated ACE2 had almost no degradation at 37° C. for 144 hours and degradation of in vivo Endo H deglycosylated ACE2 at the same condition was less than 5%. Stability assessment was further evaluated by ELISA binding study. The binding affinity study of plant produced glycosylated and deglycosylated ACE2 proteins was conducted, proteins are incubated at 37° C. for 24, 48, 72, 96, 120 and 144 hours, with commercial S protein and plant produced dRBD (FIG. 6a-d). Kd values were calculated with Graphpad Prism 5.0 software. Although the binding affinity of gACE2 and dACE2 proteins that were incubated at 37° C. for 72 or 144 hours was reduced for the commercial Spike protein, it did not change significantly for plant-produced dRBD. The difference in Kd values could be explained by several reasons such as different glycosylation status, different tags (FLAG-tagged of plant produced RBD versus His tagged of commercial insect RBD) and different amino sequences (R319-S591 of plant produced RBD versus Arg319-Phe541 of commercial insect RBD) plant produced and commercial insect RBD.

Notably, although baculovirus-insect cell system is limited by its inability to produce complex N-glycans, however, recombinant proteins produced in some insect cell lines, may contain core α1,3-linked fucose residues. Thus, based on SDS-PAGE and ELISA data, it can be concluded that plant-produced glycosylated and deglycosylated ACE2s are stable at elevated temperatures for prolonged periods of time.

Anti-SARS-CoV2 activity of plant produced glycosylated and deglycosylated forms were evaluated as seen in FIG. 7 which demonstrates apparent neutralization activities of plant produced recombinant truncated gACE2 and dACE2 variants against authentic SARS-CoV-2 in the pre-infection phase. The half maximal inhibitory concentration (IC50) values for glycosylated and deglycosylated ACE2 were 1.020 and 1.342 μg/ml, respectively, when they were mixed with 100TCID50 of SARS-CoV-2. It should be noted that in the test, the highest concentration (6 μg/ml) of gACE2 or dACE2, was non-toxic to cells.

A number of studies in the prior art have shown that a recombinant ACE2 can be used as a potential therapeutic tool in COVID-19 patients. At this point, the development and production of recombinant ACE2 protein at high levels with high anti SARS-CoV-2 activity could be a challenging task. In the present invention, it is shown that recombinant ACE2 exhibits a potent anti-SARS-CoV-2 activity with the IC₅₀ values of 1.020 μg/ml, can be produced rapidly, at high level (˜0.75 g/kg plant leaf) in N. benthamiana plant using plant transient expression system. The method and the vector of present invention demonstrates that plant produced ACEs are a cost effective, safe and promising therapeutic tool for the treatment of COVID-19 patients.

### REFERENCES

1. Fehr A R, Perlman S (2015). “Coronaviruses: an overview of their replication and pathogenesis”. Coronaviruses. Methods in Molecular Biology. 1282. Springer New York. pp. 1-23. doi:10.1007/978-1-4939-2438-7_1. ISBN 978-1-4939-2437-0.

2. Kuba K, Imai Y, Rao S, Gao H, Guo F, Guan B, et al. (August 2005). “A crucial role of angiotensin converting enzyme 2 (ACE2) in SARS coronavirus-induced lung injury”. Nature Medicine. 11 (8): 875-9. doi:10.1038/nm1267. PMC 7095783. PMID 16007097.

3. Zhou P, Yang X L, Wang X G, Hu B, Zhang L, Zhang W, et al. (March 2020). “A pneumonia outbreak associated with a new coronavirus of probable bat origin”. Nature. 579 (7798): 270-273.doi:10.1038/s41586-020-2012-7. PMC 7095418. PMID 32015507.

4. Xu X, Chen P, Wang J, Feng J, Zhou H, Li X, et al. (March 2020). “Evolution of the novel coronavirus from the ongoing Wuhan outbreak and modeling of its spike protein for risk of human transmission”. Science China. Life Sciences. 63 (3): 457-460. doi:10.1007/s11427-020-1637-5. PMC 7089049. PMID 32009228.

5. Lewis R (2020-02-20). “COVID-19 Vaccine Will Close in on the Spikes”. DNA Science Blog. Public Library of Science. Archived from the original on 2020-02-22. Retrieved 2020-02-22.

6. Li, W., Moore, M. J., Vasilieva, N., Sui, J., Wong, S. K., Berne, M. A., Farzan, M. (2003). Angiotensin-converting enzyme 2 is a functional receptor for the SARS coronavirus. Nature, 426(6965), 450-454. doi:10.1038/nature02145

7. Chan K K, Dorosky D, Sharma P, Abbasi S A, Dye J M, Kranz D M, Herbert A S, Procko E. Engineering human ACE2 to optimize binding to the spike protein of SARS coronavirus 2. Science. 2020 Sep. 4; 369(6508):1261-1265. doi: 10.1126/science.abc0870.

8. Imai Y, Kuba K, Rao S, Huan Y, Guo F, Guan B, Yang P, Sarao R, Wada T, Leong-Poi H, Crackower M A, Fukamizu A, Hui C C, Hein L, Uhlig S, Slutsky A S, Jiang C, Penniger J M (2005) Angiotensin-converting enzyme 2 protects from severe acute lung failure. Nature 436:112-116

9. Tarlan Mamedov, Ananya Ghosh, R. Mark Jones, Vadim Mett et al., Production of non-glycosylated recombinan proteins in Nicotiana benthamiana plants by co-expressing bacterial PNGase F. Plant Biotechnology Journal, 2012, 10(7):773-82.

10. Mamedov T, Cicek K, Gulec B, Ungar R, Hasanova G. In vivo production of non-glycosylated recombinant proteins in Nicotiana benthamiana plants by co-expression with Endo-β-N-acetylglucosaminidase H (Endo H) of Streptomyces plicatus. PLoS One. 2017 Aug. 21; 12(8):e0183589. doi: 10.1371/journal.pone.0183589. eCollection 2017.

11. Marnedov T, Musayeva I, Acsora R, Gun N, Gulec B, Mammadova G, Cicek K, Hasanova G. Engineering, and production of functionally active human Furin in N. benthamiana plant: In vivo post-translational processing of target proteins by Furin in plants. PLoS One. 2019a Mar. 12; 14(3): e0213438. doi: 10.1371/journal.pone.0213438. eCollection 2019.

12. Mamedov, T., Cicek, K., Miura, K., Gulec, B., Akinci, E., Mammadova, G. Z., & Hasanova, G. J. (2019). A Plant-Produced in vivo deglycosylated full-length Pfs48/45 as a Transmission-Blocking Vaccine Candidate against malaria. Scientific Reports, 9.

13. Shil P K, et al. Oral delivery of ACE2/Ang-(1-7) bioencapsulated in plant cells protects against experimental uveitis and autoimmune uveoretinitis. Mol Ther. 2014.

14. Mamedov T, Yuksel D, Ilgin M, Gurbuzaslan I, Gulec B, Yetiskin H, Uygut M A, Islam Pavel S T, Ozdarendeli A, Mammadova G, Say D, Hasanova G (2021). Plant-Produced Glycosylated and In Vivo Deglycosylated Receptor Binding Domain Proteins of SARS-CoV-2 Induce Potent Neutralizing Responses in Mice. 13(8):1595. https://doi.org/10.3390/v13081595.

15. Zhang, H., Baker, A. (2017). Recombinant human ACE2: acing out angiotensin II in ARDS therapy. Crit Care 21, 305. doi: 10.1186/s13054-017-1882-z

16. Wösten-van Asperen, R. M., Lutter, R., Specht, P. A., Moll, G. N., Van Woensel, J. B., Van der Loos, C. M., et al. (2011). Acute respiratory distress syndrome leads to reduced ratio of ACE/ACE2 activities and is prevented by angiotensin-(1-7) or an angiotensin II receptor antagonist. The Journal of pathology. 225, 618-627. doi: 10.1002/path.2987

17. Roshanravan, N., Ghaffari, S., and Hedayati, M. (2020). Angiotensin converting enzyme-2 as therapeutic target in COVID-19. Diabetes and metabolic syndrome.14, 637-639. doi: 10.1016/j.dsx.2020.05.022

18. Colafella, K. M., Bovée, D. M., and Danser, A. (2019). The renin-angiotensin-aldosterone system and its therapeutic targets. Experimental eye research.186. doi: 10.1016/j.exer.2019.05.020

19. Mamedov, T., Chichester, J. A., Jones, R. M., Ghosh, A., Coffin, M. V., Herschbach, K., Prokhnevsky, A. I., Streatfield, S. J., & Yusibov, V. (2016). Production of Functionally Active and Immunogenic Non-Glycosylated Protective Antigen from Bacillus anthracis in Nicotiana benthamiana by Co-Expression with Peptide-N-Glycosidase F (PNGase F) of Flavobacterium meningosepticum. PloS one, 11(4), e0153956. https://doi.org/10.1371/journal.pone.0153956

20. Mamedov T, Yuksel D, Ilgin M, Gürbüzaslan I, Gulec B, Mammadova G, Ozdarendeli A, Yetiskin H, Kaplan B, Islam Pavel S T, Uygut M A, Hasanova G (2021). Production and Characterization of Nucleocapsid and RBD Cocktail Antigens of SARS-CoV-2 in Nicotiana benthamiana Plant as a Vaccine Candidate against COVID-19, 9(11):1337. https://doi.org/10.3390/vaccines9111337

1. A method for generating a polypeptide of glycosylated Angiotensin-converting enzyme 2 (ACE2) in a plant cell, characterized by comprising the steps of:
i. replacing signal peptide of human ACE2 with Nicotiana tabacum PR-1a signal peptide having amino acid sequence of SEQ ID NO.7, adding ER retention signal having amino acid sequence of SEQ ID NO.6 and adding His6 tag coding sequence to C-terminus and constructing an artificial ACE2 gene; wherein the artificial ACE2 gene is operable linked to a promoter such that when the promoter is activated, the ACE2 polypeptide is expressed,
ii. inserting the constructed ACE2 gene into small binary vector tailored for transient expression (pEAQ vector) to obtain pEAQ-ACE2-His6-KDEL plasmid having a nucleotide sequence that has at least 90 percent sequence identity to sequence of SEQ ID NO:1,
iii. introducing pEAQ-ACE2-His6-KDEL plasmid having a nucleotide sequence that has at least 90 percent sequence identity to sequence of SEQ ID NO:1 into an Agrobacterium construct,
iv. performing infiltration of the Agrobacterium construct carrying the pEAQ-ACE2-His6-KDEL plasmid having a nucleotide sequence that has at least 90 percent sequence identity to sequence of SEQ ID NO:1 into a plant cell and producing a polypeptide of glycosylated ACE2 having amino acid sequence of SEQ ID NO.2.

2. A method for generating a N-deglycosylated polypeptide of ACE2 in a plant cell, characterized by comprising the steps of:
replacing signal peptide of human ACE2 with Nicotiana tabacum PR-1a signal peptide having amino acid sequence of SEQ ID NO.7, adding ER retention signal having amino acid sequence of SEQ ID NO.6 and adding His6 tag coding sequence to C-terminus and constructing an artificial ACE2 gene,
inserting the constructed ACE2 gene into small binary vector tailored for transient expression (pEAQ vector) to obtain pEAQ-ACE2-His6-KDEL plasmid having a nucleotide sequence that has at least 90 percent sequence identity to sequence of SEQ ID NO:1; wherein the artificial ACE2 gene is operable linked to a promoter such that when the promoter is activated, the ACE2 polypeptide having amino sequence of SEQ ID NO.2 is expressed,
separately from pEAQ-ACE2-His6-KDEL plasmid, constructing an ENDO H-Flag-KDEL plasmid having nucleotide sequence of SEQ ID NO.4 by adding a second nucleic acid encoding a bacterial Endo-β-N-acetylglucosaminidase H (Endo H), adding ER retention signal having amino acid sequence of SEQ ID NO.6 and adding Flag tag coding sequence; wherein the Endo H sequence is operable linked to a promoter such that when the promoter is activated, the Endo H polypeptide having amino acid sequence of SEQ ID NO.5 is expressed,
introducing pEAQ-ACE2-His6-KDEL plasmid having a nucleotide sequence that has at least 90 percent sequence identity to sequence of SEQ ID NO:1 into the Agrobacterium construct,
introducing ENDO H-Flag-KDEL plasmid having nucleotide sequence of SEQ ID NO.4 into another Agrobacterium construct,
performing co-infiltration of Agrobacterium construct carrying the pEAQ-ACE2-His6-KDEL plasmid having a nucleotide sequence that has at least 90 percent sequence identity to sequence of SEQ ID NO:1 with Agrobacterium construct carrying ENDO H-Flag-KDEL plasmid having nucleotide sequence of SEQ ID NO.4 into a plant cell and producing a polypeptide of deglycosylated ACE2 having amino acid sequence of SEQ ID NO.2; wherein by action of the Endo H polypeptide having amino acid sequence of SEQ ID NO.5, ACE2 is deglycosylated with no amino acid change in the asparagine-X-serine/threonine (NXS/T) site, wherein X is any amino acid except proline of resulting 15 polypeptide, opposite to that of action of the bacterial PNGase F which causes amino acid change in the deglycosylated protein targets due to deamidation of the asparagine (N) in the NXS/T site (sequence) into an aspartate (D).

3. The method according to claim 1 or claim 2, wherein the Agrobacterium is Agrobacterium tumefaciens strain AGL1.

4. The method of according to claim 1 or claim 2, wherein the plant cell is a Nicotiana benthamiana leaf cell.

5. The method of according to claim 4, wherein the Nicotiana benthamiana is 6-7-week-old Nicotiana benthamiana.

6. A glycosylated Angiotensin-converting enzyme 2 (ACE2) polypeptide produced in a plant cell by the method according to claim 1.

7. The glycosylated ACE2 polypeptide according to claim 6, wherein the plant cell is a Nicotiana benthamiana leaf cell.

8. A N-deglycosylated Angiotensin-converting enzyme 2 (ACE2) polypeptide produced in a plant cell by the method according to claim 2.

9. The N-deglycosylated ACE2 polypeptide according to claim 8, wherein the plant cell is a Nicotiana benthamiana leaf cell.

10. A truncated Angiotensin-converting enzyme 2 (ACE2) protein generated in a plant cell for use in treatment of COVID-19, characterized by comprising an amino acid sequence of SEQ ID NO:2.

11. The truncated ACE2 protein according to claim 10, wherein the plant cell is a Nicotiana benthamiana leaf cell.

12. A vector for generating the truncated ACE2 protein according to claim 10 or claim 11, characterized by comprising a nucleic acid sequence that has at least 90 percent sequence identity to the sequence of SEQ ID NO:1 encoding ACE2 gene, wherein the nucleic acid sequence is operable linked to a promoter such that when the promoter is activated, the ACE2 polypeptide is expressed.

13. A product for use in treatment of COVID-19, characterized by comprising a glycosylated Angiotensin-converting enzyme 2 (ACE2) protein having an amino acid sequence of SEQ ID NO:2.

14. A product for use in treatment of COVID-19, characterized by comprising a deglycosylated Angiotensin-converting enzyme 2 (ACE2) protein that produced by co-expression of Endo H with the expression of ACE2, having an amino acid sequence of SEQ ID NO:2.
